(19) United States
(12) Patent Application Publication
(10) Pub. No.: US 2025/0064077 A1
(43) Pub. Date: Feb. 27, 2025

(54) A METHOD AND APPARATUS FOR THE PREPARATION OF A BEVERAGE

(71) Applicant: Koninklijke Douwe Egberts B.V., Utrecht (NL)

(72) Inventors: Fabien Guilmineau, Banbury (GB); Willem Plantinga, Utrecht (NL); Alexandr Kachko, Utrecht (NL); Hendrik Joan Dees, Utrecht (NL)

(21) Appl. No.: 18/725,088

(22) PCT Filed: Dec. 22, 2022
(86) PCT No.: PCT/EP2022/087599

§ 371 Date: Jun. 27, 2024

(30) Foreign Application Priority Data
Dec. 31, 2021 (GB) 2119150.7

(51) Int. Cl. A23F 5/14; A23F 5/40; A47J 31/42; A47J 31/52; G01N 21/3563; G01N 21/359; G01N 33/14

(52) U.S. Cl. CPC A23F 5/145; A23F 5/40; A47J 31/42; A47J 31/52; G01N 21/3563; G01N 21/359; G01N 33/14

(57) ABSTRACT

This disclosure provides a method of determining the brewing parameters for preparing a coffee beverage with a coffee brewing apparatus, comprising: receiving, at a sensing arrangement, a coffee product for brewing a coffee beverage; obtaining, with the sensing arrangement, a characteristic of the coffee product; using the characteristic to determine one or more brewing parameter for the coffee product and brewing a coffee beverage using the one or more brewing parameter.

### TECHNICAL FIELD OF THE INVENTION

The present disclosure relates to a method and apparatus for preparing a coffee beverage, and a method for preparing a coffee product for use in a coffee brewing apparatus. In particular, the methods and apparatus seek to enable the identification of a coffee product for use in the preparation of a coffee beverage in a coffee brewing apparatus.

### BACKGROUND TO THE INVENTION

Coffee brewing apparatuses which prepare and dispense coffee beverages are well known in the art. Many of these known machines include the option to adjust one or more settings in accordance with a particular variety of coffee. For example, a coffee brewing machine may vary one or more brewing parameters in dependence of the type of coffee used, the country of origin or the manufacturer of the coffee. However, the adjustment of parameters requires manual intervention of a user, often on a trial or error basis. This is time consuming, subjective, and often inconsistent, which are generally undesirable.

The present invention seeks to provide an improved method and apparatus for brewing a coffee beverage, and also seeks to provide a method of preparing a coffee product which can aid identification of the coffee product.

### SUMMARY OF THE INVENTION

The present invention provides a method and apparatus according to the appended claims.

The present disclosure provides a method of determining the brewing parameters for preparing a coffee beverage with a coffee brewing apparatus. The method may comprise: receiving, at a sensing arrangement, a coffee product for brewing a coffee beverage; obtaining, with the sensing arrangement, a characteristic of the coffee product; and, using the characteristic to determine one or more brewing parameter for the coffee product. The method may further comprise configuring the coffee brewing apparatus to brew the coffee beverage using the determined one or more brewing parameter.

Providing a coffee product which can be sensed by a sensing arrangement of a coffee brewing apparatus to obtain one or more characteristics of the coffee product and using this characteristic to determine the brewing parameters allows the brewing parameters to be determined on the basis of the coffee product alone. This allows for a more consistent setting of the brewing parameters in accordance with a coffee type, origin or manufacturer or the like.

Further, the method of the present disclosure also facilitates the automation of the coffee brewing apparatus such that the brewing parameters can be automatically set in dependence of a coffee product which is loaded into the brewing apparatus. Hence, there is less requirement for user input which improves the convenience of the coffee brewing apparatus.

The coffee product may comprise a marker, preferably a marker added or applied to the coffee product. In preferred embodiments the coffee product comprises an added or applied marker and the characteristic relates to the marker.

The method may therefore comprise a method of determining the brewing parameters for preparing a coffee beverage with a coffee brewing apparatus, comprising:
receiving, at a sensing arrangement, a coffee product with an applied or added marker, for brewing a coffee beverage;
obtaining, with the sensing arrangement, a characteristic of the marker applied or added to the coffee product; and,
using the characteristic to determine one or more brewing parameter for the coffee product.

The marker may be configured to allow the coffee product to be discriminated from other coffee products and/or to allow the coffee product to be more readily identified and the brewing parameters selected. The characteristic obtained by the sensing apparatus may comprise a characteristic of the marker and/or a characteristic of the marker in combination with coffee beans within the coffee product.

The coffee product may comprise coffee beans. The coffee beans may be roasted whole coffee beans. The marker may comprise one or more of the group consisting: a surface coating of at least one coffee bean and an instant coffee. Thus, the coffee product may comprise a blend of an instant coffee, e.g. a powdered coffee, and/or coffee beans which have a surface coating of a substance which is detectable by the sensing arrangement.

The marker may be consumable such that it can form part of the coffee beverage when the coffee beans are brewed. When the marker comprises one or more surface coating, the surface coating is preferably organic or a salt. The organic coating may comprise a coffee-based substance. Providing an instant coffee of a coffee-based substance for a surface coating allows the brewed coffee beverage to comprise coffee only. This may be preferable for a consumer of the coffee beverage.

The coating may comprise a residue from a water based solution which is applied to roasted coffee beans. The water based solution may be applied as a quenching agent following a roasting process or applied to roasted and quenched or otherwise cooled coffee beans. The organic coating may comprise one or more from the group comprising: a high temperature extract; a tertiary extract; a coffee cherry extract; a green coffee extract; an instant coffee extract; and silverskin extract. The instant coffee and coffee-based coating may comprise coffee of the same variety, origin and/or manufacturer of the coffee beans.

The sensing arrangement may be configured to detect one or more substances or molecules within the marker. The substances or molecules may comprise simple sugars, native chlorogenic acid; manno-oligosaccharides; galactomannans; an antioxidant, calcium, magnesium, potassium and chloride.

Where the marker comprises a salt, the salt may comprise one or more of the group consisting of: calcium dichloride; magnesium dichloride; potassium hydroxide and potassium chloride.

The surface coating may comprise a residue from a quenching agent used to quench the coffee beans following roasting.

The sensing arrangement may be configured to obtain spectroscopic data from the coffee product. The spectroscopic data may comprise visible light or near infrared spectroscopic data. The method may further comprise illuminating the coffee product with electromagnetic waves of a predetermined wavelength, e.g. near infra-red, detecting the reflected electromagnetic waves and storing the spectroscopic data of the detected electromagnetic waves. The spectroscopic data may comprise intensity data for a plurality of wavelengths. The wavelengths may range between 1100 nm and 2500 nm.

The brewing parameter may be any brewing parameter which affect the quality of the brewed coffee. The brewing parameters may relate to one or more controllable parameters of the coffee brewing apparatus. The brewing parameters may comprise one or more of water temperature, grain size of the ground coffee product, water flow rate, water pressure, brew volume, particle size distribution, type of grinder, compaction amount, volume of coffee dispensed to the coffee brewer and/or brewing time. Thus, the brewing parameter may be associated with a grinder of the coffee brewing apparatus, a doser, a water heater of the brewing apparatus, or a brewing chamber of the brewing apparatus.

The coffee product may be stored within a hopper of the coffee brewing apparatus. The sensing arrangement may be located proximate to the hopper such that the coffee product can be sensed whilst in the hopper. For example, the sensing arrangement may sense the coffee product through a wall of the hopper.

The coffee product may have a predetermined moisture value. The moisture value may be controlled using a quenching solution and/or a marker solution which is applied to the cooled coffee beans following roasting and, optionally, quenching. The coffee product may comprise a moisture value between 3% and 5% w/w.

The coating of the coffee product which provides the marker may be required to have a minimum weight of solids so as to be detectable by the sensing arrangement. In some embodiments, the coating may comprise between 0.5% and 1.5% w/w solids.

The coffee beverage may comprise a solids content of the coating of less than 3% of the nutritional recommended value per 100 ml for the solid. As such, the marker may be retained with the prepared coffee beverage whilst being within the nutritional recommended values. This may be reassuring for a consumer and generally preferable from a regulatory viewpoint.

The present disclosure may further relate to the preparation of a coffee product or use in a coffee brewing apparatus. The method may further comprise: preparing coffee beans for use in a coffee beverage apparatus, comprising: roasting green coffee beans in a roaster to provide roasted coffee beans; and, applying a marker to the roasted coffee beans to reduce the temperature of the coffee beans; and, providing the coffee beans to a coffee beverage apparatus. The marker may be provided as a residue or a quenching agent or a marking solution applied to roasted, cooled coffee beans.

The method of preparing the coffee product may further comprise: determining the moisture level of roasted coffee beans following quenching; preparing a water based marking solution having a predetermined amount of marker, wherein the applying the marker to the coffee beans comprises applying the marking solution, and wherein the amount of marker within the marking solution is determined based on the measured moisture level, a required final moisture level of the roasted coffee beans and a required amount of marker residue.

The coffee product may comprise a moisture value between 3% and 5% w/w. The coating may comprise between 0.5% and 1.5% w/w solids.

The prepared coffee may comprise a solids content of the coating of less than 3% of the nutritional recommended value per 100 ml for the solid.

The present disclosure provides a coffee brewing apparatus configured to carry out any of the methods disclosed herein.

The coffee brewing apparatus may comprise: a hopper for storing/containing a coffee product; a sensing arrangement configured to obtain a characteristic of the coffee product; and, a controller configured to receive an input from the sensing arrangement, the input relating to the characteristic of the coffee product. The controller may be configured to determine one or more brewing parameters using the characteristic, and control the coffee brewing apparatus to brew a coffee beverage using the one or more brewing parameters.

The sensing arrangement may comprise a spectrometer. The spectrometer May comprise a near infrared spectrometer.

The apparatus may further comprise a coffee grinder. The one or more brewing parameters may comprise a parameter of the grinder.

The apparatus may comprise a brewing chamber and a water heater. The controller may be configured to control the water heater to control the water temperature according to the determined brewing parameter. The controller may be configured to control the water flow rate and/or brewing time of the coffee product according to the determined brewing parameters.

The coffee product may comprise roasted whole beans and an instant coffee.

The coffee brewing apparatus may comprise a computer memory configured to store a look-up table of known characteristics of a known coffee product, wherein the known characteristics for the known coffee product are associated with one or more predetermined brewing parameters. The one or more predetermined brewing parameters may be ideal brewing parameters for the known coffee product. The controller may be configured to compare the obtained one or more characteristics with the known characteristics to identify the appropriate brewing parameters for the coffee product.

The present disclosure further provides a method of determining brewing parameters of a coffee product, wherein the coffee product comprises coffee beans and an instant coffee, the method comprising: obtaining spectroscopic data of the coffee beans and instant coffee; comparing the spectroscopic data to known spectroscopic data, the known spectroscopic data being associated brewing parameters; and, using the associated brewing parameters as the brewing parameters for the coffee product when the spectroscopic data and known spectroscopic data are comparable. That is, the associated brewing parameters are used as the brewing parameters when the spectroscopic data and the known spectroscopic data are the same or similar.

The present disclosure further provides a coffee product comprising a plurality of coffee beans and a marker. The marker may be a coating on the coffee beans or an instant coffee. When the marker is a coating, the marker may be a coffee based marker or a salt. The skilled person will appreciate that except where mutually exclusive, a feature described in relation to any one of the aspects, embodiments or examples described herein may be applied mutatis mutandis to any other aspect, embodiment or example. Furthermore, except where mutually exclusive, any feature described herein may be applied to any aspect and/or combined with any other feature described herein.

### BRIEF DESCRIPTION OF THE DRAWINGS

In order that the invention may be more clearly understood one or more embodiments thereof will now be described, by way of example only, with reference to the accompanying drawings, of which:

FIG. 1 shows a method of determining one or more brewing parameters for preparing a coffee beverage using a coffee brewing apparatus according to the present disclosure;

FIG. 2 shows a method of preparing a coffee product according to the present disclosure;

FIG. 3 shows spectroscopic data for a plurality of coffee products;

FIG. 4 shows a principal component analysis, PCA, map for a plurality of samples of the coffee product spectroscopic data shown in FIG. 3;

FIG. 5 shows a PCA map for coated coffee beans and reference coffee beans;

FIG. 6 shows a PCA map of alternative coated coffee beans and reference beans;

FIG. 7 shows a PCA map for a plurality of coated coffee beans in which the coating comprises a salt;

FIG. 8 shows a combined PCA map for coated coffee beans having a salt based coating, a coffee based coating and a roasted whole bean and instant coffee blend;

FIG. 9 shows a coffee brewing apparatus which may be utilised for carrying out one or more of the methods disclosed herein; and,

FIG. 10 shows a method of operating the coffee brewing apparatus of FIG. 9.

### DETAILED DESCRIPTION OF THE INVENTION

### General Overview of the Method

In the following detailed description, numerous specific details are set forth in order to provide a thorough understanding of various embodiments and the inventive concept. However, those skilled in the art will understand that the present invention may be practiced without these specific details or with known equivalents of these specific details, that the present invention is not limited to the described embodiments, and that the present invention may be practiced in a variety of alternative embodiments. It will also be appreciated that well known methods, procedures, components, and systems may have not been described in detail.

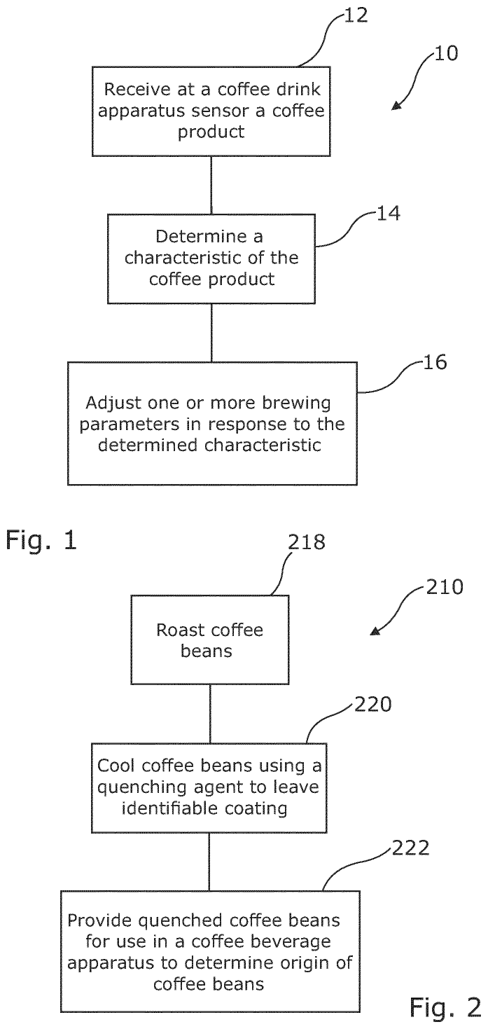

FIG. 1 shows a flow diagram for a method 10 of preparing a coffee beverage according to the present disclosure. The method 10 comprises: receiving 12 at a coffee brewing apparatus sensing arrangement a coffee product; determining a characteristic of the coffee product 14 using the sensing arrangement; and, adjusting one or more brewing parameters 16 in response to the determined characteristics.

The method of the present disclosure allows a coffee product to be analysed in order to determine the characteristics of the coffee product and, in response to the determined characteristics, adjust how the coffee product is brewed. In doing so, the brewing apparatus may be specifically configured to brew a particular type or condition (e.g. moisture content) of a coffee bean, thereby improving the quality of the brewed coffee beverage. In addition, or alternatively, analysing the beans and making associated adjustments to the brewing parameters may allow the brewed beverage to be prepared more consistently. As such, the consumer of the beverage may receive a more consistent quality of coffee beverage which ultimately increases the consumer experience.

An advantage of providing a sensing arrangement which is configured to determine a characteristic of a coffee product within the coffee brewing apparatus is that it allows the coffee brewing apparatus to be automatically adjusted in response to the sensed characteristics of the coffee product. Thus, a coffee brewing apparatus may be configured to sense and adjust the brewing parameters automatically and in response, for example, to a new coffee product being loaded into the apparatus or when prompted to by a user or under certain conditions (such as when powered up or following a cleaning cycle).

It will be appreciated that the sensing of the coffee-product typically comprises the direct sensing of the coffee product rather than indirect sensing. Thus, in some embodiments of the present disclosure, the coffee product may be exposed to an electromagnetic wave radiation and undergo some form of spectroscopic analysis to determine the characteristics. An indirect method of determining a characteristic of the coffee product may be achieved with the use of a labelling or marking on the coffee product packaging. For example, the packaging may include one or more barcode or QR code which a user of the coffee brewing apparatus may scan with the apparatus when loading the coffee product. It will be appreciated that an indirect sensing is less advantageous as it requires the user to locate and scan the label on the package and is prone to both user error and errors or scanning difficulties in relation to the packaging. Directly analysing the coffee product provides more accurate and reliable results which are more readily obtained.

The characteristic of the coffee product may relate to one or more of the non-exhaustive group comprising: a manufacturer, a bean variety or species, and the country of origin of the bean. The characteristic may also indicative of other attributes of the coffee bean such as moisture content of the coffee beans. In short, the characteristic may be indicative of any attribute of the bean which may affect the required brewing parameters. The characteristic can be matched to a corresponding characteristic of known coffee products having known preferred brewing parameters, thereby allowing the brewing parameters of the analysed coffee product to be determined.

The characteristic may be within the data outputted by a sensor, e.g. a detector, of the sensing arrangement. Hence, the signal generated by the sensing arrangement may be encoded with information which represents a characteristic of the coffee product. Hence, for example, the sensor arrangement may comprise a spectrometer having a detector with the signal outputted from the detector including spectral information relating to the coffee product, with features within the spectral information providing characteristics of the coffee product. Hence, the characteristics may, in some embodiments, be referred to as spectral characteristics.

Comparing the obtained spectral characteristics to spectral characteristics of known coffee products having known brewing parameters can reveal which brewing parameters may be preferred for the analysed coffee product. Hence,
once a match between the obtained spectral information and that of a known coffee product has been made, the brewing parameters can be adjusted accordingly. It will be appreciated that the “obtained” spectral characteristics may be measured or detected spectral characteristics, as described herein.

### Coffee Product

The term “coffee product” may be understood to include any coffee-based substance which is used within the coffee brewing apparatus to make a coffee beverage. The coffee product may comprise only coffee or coffee-based substances. Providing a coffee product which comprises only coffee or coffee-based substances allows the resultant coffee beverage to contain only coffee and removes the need for artificial or non-coffee derived additives. This may be desirable for a consumer who only wishes to have coffee-based substances within the coffee beverage and to avoid having additional ingredients.

In some embodiments, the coffee product may comprise one or more of a coffee bean and an instant coffee, ground coffee or powdered coffee. References to instant coffee herein may be taken to include ground coffee and powdered coffee.

Where the coffee product comprises coffee beans, the coffee beans will typically be roasted whole beans, RWB, which are provided prior to grinding. Hence, in many embodiments, the coffee brewing apparatus will comprise a grinding apparatus. The grinding apparatus may comprise one or more adjustable settings which represent one or more brewing parameters. Thus, the grinding apparatus may be configured in response to a sensed or determined characteristic of the coffee product and the desired brewing parameter for that coffee product. The grinding parameters may comprise the grind size or other parameters where applicable, such as the type of grinder, grinding speed and instant particle size distribution. These parameters may impact the quality of the coffee and/or the brewing time, water temperature or flow rate of the water used in the coffee brewer, as well known in the art.

The coffee beans may be a single type and/or from a single source. However, this is not a limitation and a blend of coffee beans may be used. The instant coffee may be of the same type and/or from the same source as the coffee beans, however, this is not a limitation and it may be possible to use specific types of instant coffee to provide a suitable marker and aid improved identification.

The coffee beans may include one or more coating. The coating may be a residual coating left over from a manufacturing process used to prepare the coffee beans, or a coating which is purposely deposited on the beans to provide a marker specifically for identification purposes. The coating may be introduced during the preparation of the coffee beans as part of a conventional manufacturing step. For example, the coating may be residue of a quenching agent used to cool the beans following roasting. In other embodiments, the coating may be applied following a roasting and quenching process.

The use of quenching water to cool roasted coffee beans is well known in the art and allows the beans to be cooled more effectively and efficiently than using air. More rapid quenching helps to avoid over-roasting and help halt exothermic reactions within the beans. Quenching is typically carried out using water which may be sprayed or poured onto the beans, or used for immersion of the beans.

The method of the present disclosure may include quenching agents which may comprise one or more substances, such as organic compounds or salts, which leave a residue, i.e. a coating, on the coffee beans. The coating may be selected to provide a specific signature in the spectral data as a characteristic of the coffee bean for discrimination purposes.

As shown in FIG. 2, the present disclosure provides a method preparing coffee beans 210 wherein the coffee beans are roasted 218 and quenched 220 using a quenching agent or other solution. The quenching agent or other solution comprises one or more substances that leave an identifiable residue, i.e. a coating, on the beans. The resultant marked beans may then be used 222 in a coffee brewing apparatus in which the coffee beans can be analysed and the coating used to provide a characteristic of the coffee product and the required associated brewing parameters.

Typically, the coating will be a consumable substance which can be incorporated into the coffee beverage. As noted above, the coating may comprise one or more organic compounds or salts. The organic compound may comprise a coffee-based substance such that the brewed coffee beverage only comprises only coffee and water. Where the coating comprises salts, the salts may be provided only in small amounts such that they can be safely consumed as part of the brewed coffee beverage.

The quenching agent or other solution may comprise any form of coffee extract including, for example, a cherry extract, a green coffee extract, an instant coffee extract or a silverskin extract. The coffee extract may be a tertiary extract and/or a high temperature extract carried out at a temperature of between 200 and 260 degrees C. By tertiary extract it is meant an extract taken from previously extracted spent coffee grounds. Hence, a primary extract may comprise using roast and ground beans and extracting the coffee to produce a primary extract and spent coffee grounds. A secondary extract may be produced from the spent coffee grounds in a second extraction, usually at a higher temp than the first extraction. The tertiary extract may comprise using the spent coffee grounds from the secondary extraction process and undertaking a third extraction at an even higher temperature. As noted, the tertiary extraction may be carried out at 200-260 degrees C. for example. In some embodiments, the quenching agent may comprise one or more salt solutions comprising one or more from the group comprising: calcium dichloride; magnesium dichloride; potassium hydroxide or potassium chloride. A summary of some example coatings and the associated detectable marker is provided in Table 1, below.

TABLE 1
coating options for the purpose of marking
a coffee bean for identification purposes.

| Marking option | Source of marker | Molecules/marker |
|---|---|---|
| 1. Coating the surface of | Cherry extract | Simple sugars |
| the beans with a water- | Green Coffee extract | Native CGA |
| based solution of coffee | Silverskin extract | Anti-oxidant |
| based material, as a thin | Instant coffee extract | Galactomannans |
| coat after cooling | | |
| 2. Coating the surface of | CaCl2 solution | Calcium |
| the beans with a water- | MgCl2 solution | Magnesium |
| based mineral salt solution | KOH solution | Potassium |
| to adjust the mineral | KCl solution | Chloride |
| composition of the quench | | |
| water | | |

The origin of the quenching agent or other solution additive may be any suitable source as known in the art. In the case of a cherry extract, this may be obtained from the cascara of the coffee cherries once the beans have been removed and using a hot water extraction method. A green coffee extract may be obtained using a hot water extraction from a ground green coffee bean. The silverskin extract may be obtained from a hot water extraction process using chaff from, for example, a roaster. The salts may be available commercially or prepared according to well known methods.

The use of the term “or other solution” refers to embodiments in which the coating is applied outside of the quenching process, for example, where the beans are cooled. Generally, for the purpose of the present disclosure, the term “quenching agent” may be considered to be any solution which can be applied to the beans whether or not as part of a quenching process.

Where the coffee product comprises an instant coffee, the instant coffee may be a powdered coffee and/or may be obtained from coffee beans that have been roasted, ground and brewed with water to form a liquid (aqueous) extract which is then dehydrated or concentrated. The dehydrated or concentrated instant coffee may then be dissolved or diluted as required, to form a coffee beverage. The dehydration process of the extract may be achieved by, for example, freeze-drying or spray-drying. It will be appreciated that the instant coffee will ideally be the same coffee as the beans within the coffee product, but this is not a limitation and a blend of different varieties, species and origins may be possible.

### Sensing Arrangement

The characteristics of the coffee product may be determined using any known methodology. In some embodiments, the coffee product may be analysed using an electromagnetic wave radiation, such as the visible or near-infrared spectrum. As such, a portion of the coffee product may be exposed to a suitable source of illumination and the reflected light analysed to determine one or more characteristics which is indicative of the properties of the coffee product. The analysis may comprise spectroscopy, particularly near infrared spectroscopy.

The use of near infrared spectroscopy for assessing the quality and origin of coffee beans is known from Páscoa, N. R. M. J et al, Use of Near-Infrared Spectroscopy for Coffee, Coffee in Health and Disease Prevention, Academic Press, 2015, ISBN 9780124095175, pages 933 to 942; and Sarraguça, M. C. et al, Authenticity Control of Roasted Coffee Brands, Food Anal. Methods, 213 September 2012, both of which are incorporated herein by reference. These documents both focus on the analysis of unmodified green, roasted and spent coffee in bean or ground forms with a view to assessing the origin and quality of the coffee. They do not contemplate the use of near-infrared (NIR) spectroscopy to determine a characteristic of a coffee for the purpose of selecting brewing parameters nor modifying a coffee product, e.g. to include a marker, to aid the analysis of the coffee product and improve discrimination.

The spectroscopy of the present disclosure may be carried out in the visible and NIR region of electromagnetic spectrum, e.g. 400-2500 nm, with a suitable resolution, e.g. 2 nm, and data acquisition rate to enable an accurate and relatively swift analysis of the coffee product once loaded into the coffee brewing apparatus. In some embodiments, the spectroscopy may be limited to the NIR spectral range i.e. 700 nm to 2500 nm, or a more specific range of between 1100 nm and 2500 nm, or to specific ranges known to be sensitive for coffee. For example, the differences between spectra of different coffees may be manifested strongly by the wavelengths attributed to fat and protein and the analysis may be restricted to one or more of these wavelength ranges if desired. This may be advantageous for, for example, reducing the size or complexity of the sensor arrangement or the signal processing which occurs on the captured data.

The sensing arrangement may be conventional and comprise the principal components of a source for emitting the electromagnetic waves, a detector for receiving the reflected waves, and a dispersion element, such as a prism or diffraction grating or a static dispersion filter such as a linear variable filter, to allow the intensity of the different wavelengths to be recorded. The sensing arrangement may also comprise one or more processors, controllers or logic circuits configured to carry out the spectral analysis of the received data and to identify the characteristic of interest. However, the data processing may be carried out by a separate module of the brewing apparatus or elsewhere.

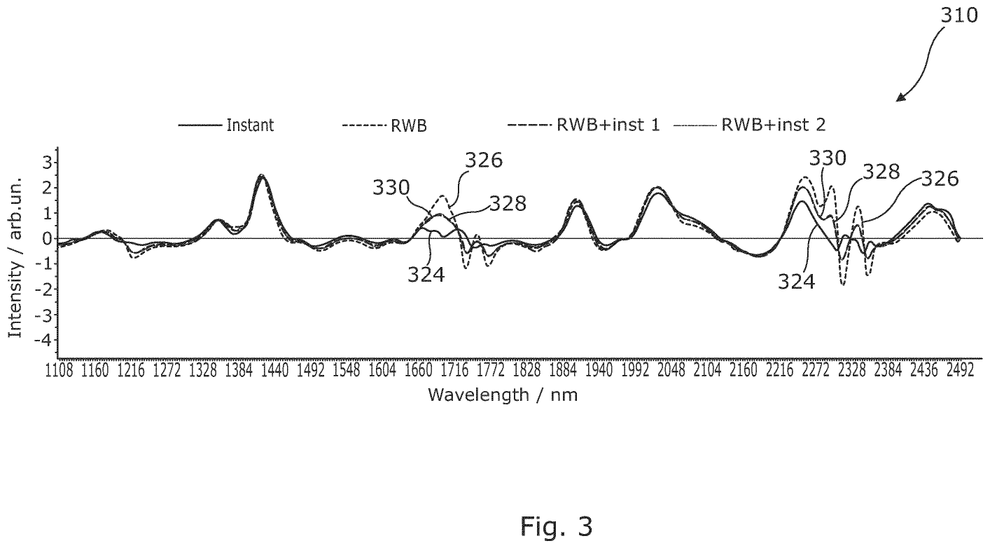

FIG. 3 shows a representation 310 of a some measured spectral data in the 1100 nm to 2500 nm wavelength range. The y-axis shows the intensity in an arbitrary unit at each wavelength with the x axis showing the wavelengths between 1108 nm and 2492 nm. There are four plots included in FIG. 3: the first corresponds to an instant coffee 324, the second corresponds to roasted whole beans 326 in the absence of instant coffee, the third corresponds to roasted whole beans mixed with instant coffee of a first grain size 328, and the fourth corresponds to roasted whole beans mixed with an instant coffee having a smaller particle size 330. Generally, although not exclusively, the instant only plot line 324 is provided closest to the x-axis with the roasted whole bean plot 326 being furthest away. The mixed bean and instant coffee plots are predominantly presented between lines 324 and 326 and largely overlapping and generally indistinguishable as shown.

### Data Processing

The spectroscopy data may be processed in any suitable way to identify one or more spectral characteristics of the coffee product. The spectral characteristic may correspond to a particular spectra, spectral features or data derived from the spectra. For example, in some embodiments the spectra may be analysed to determine one or more features which can be matched to predetermined features of known spectra of a known coffee product. The known coffee product may be associated with a manufacturer, coffee type, or country of origin, for example, and allow the measured coffee product to be matched to a known sample. For each known sample the ideal or desired brewing parameters may be selected and used for brewing the coffee.

The processing of the spectral data may comprise any suitable filtering or other pre-processing which enables the defining characteristics of the spectral data which relate to the coffee product to be obtained. For example, the pre-processing may be used to remove noise or unwanted spectral information. The pre-processing may comprise a standard normal variate transformation to reduce or remove multiplicative effects of scattering and particle size, as is well known in the art. The standard normal variate, SNV, transformation may centre and scale the individual spectrum so as to have a mean of 0 (μ=0) and a variance of 1 (σ=1), for example. The data may be further transformed by detrending to remove non-linear baselines.

In combination, SNV and detrending reduce multicollinearity, baseline shift and curvature, as disclosed in S. Verboven, S., et al, Robust pre-processing and model selection for spectral data,” J. Chemom., 2012, vol. 26, no. 6, pp. 282-289, which is incorporated herein by reference. Other pre-processing techniques, such as orthogonal signal correction, windowing or wavelet based methods may also be used.

The spectra shown in FIG. 3 have undergone a pre-processing comprising two methods. The first is a Savitzky-Golay filter (derivative order: 1, polynomial order: 2, smoothing points: 3), and second method is SNV. After the pre-processing the scatter effects due to particle size variation should be minimized with the remaining data describing mostly chemical features of samples. The differences between spectra are manifested strongly by the wavelengths attributed to fat (1160 nm, 1210 nm, 1726 nm, 2308 nm, 2354 nm) and protein (2054 nm and 2280 nm).

Following pre-processing, where carried out, the data may be analysed to identify the characteristic or characteristics using any suitable technique known in the art. One convenient method for analysing the data is principal component analysis which was found to suitably distinguish between samples of different coffee product. Principal component analysis involves representing the spectral data using only the principal components which are used to differentiate the possible different coffee products and/or match the spectral data to known coffee products. Principal component analysis is well known in the art and favourable as it provides a simple and reasonably robust way of differentiating different types of coffee product from one another, and also allows a visualisation of the result, if required.

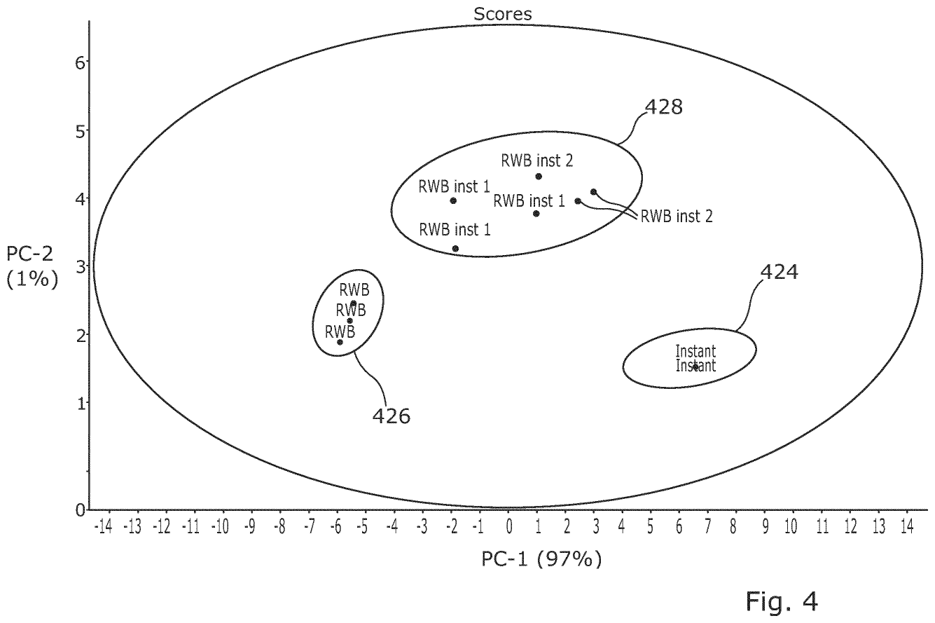

FIG. 4 shows an example of a PCA map of the spectra shown in FIG. 3 for multiple samples. As can be seen, the different types of coffee product roasted whole bean 426, instant coffee 424 and the roasted whole bean and instant coffee mixes 428 are well separated into different areas and easily distinguishable from one another.

The use of a PCA map here is used to demonstrate the effectiveness of using NIR spectroscopy and the differentiation that can be achieved. However, it will be appreciated that in a working system the use of PCA may not be required per se and the apparatus may be configured to simply adjust the parameters.

Other ways of processing the data may include: Partial Least Square analysis, Discriminant Analysis, Support Vector Machines, Convolutional Neural Networks, Recurrent Neural Networks, amongst others.

It will be appreciated that the use of NIR spectroscopy is well-known in the art and various software programs may be used within the brewing apparatus to analyse the spectral data. Suitable computer programs include, for example, Unscrambler X version 10.5 from CAMO software AS, Microsoft Excel 2016 MSO version 16.0 from Microsoft Corp., ISIscan 4.11 or WinISI 4 from Intrasoft International LLC, Python and R dedicated packages tailored to the task of coffee differentiation.

### Brewing Parameters

Once analysed, the spectral data can be compared to known spectral data to provide associated predetermined brewing parameters. The brewing parameters may comprise one or more from the group comprising: water temperature, water flow rate or pressure, brew volume, amount of roasted and ground coffee, compaction of the coffee bed, volume of coffee product, or the grind size of a grinder for example. The specific values for the brewing parameters are conventional and dependent on the particular coffee type and thus not described in detail here.

### Moisture Levels

It will be appreciated that, in some embodiments, the coating, which may be provided as a quenching agent or otherwise, may comprise the marker for coating the beans. Whether a quenching agent which includes the marker or a separate coating solution is used, it is generally desirable to ensure that the amount of the coating which is deposited on the beans is sufficient to allow identification.

In order to provide a suitable coating of the marker, it is desirable to determine the amount of the coating substance within the solution in accordance with the moisture levels of the roasted beans and a target moisture level. In doing so, it is possible to ensure that the moisture level in the prepared marked coffee product is as desired for grinding and brewing, with the necessary amount of coating to allow for identification of the coffee product.

In some embodiments, the roasted beans may undergo a quench using a minimal amount of water. The initial quench may be sufficient to reduce the temperature in the beans and prevent any exothermic reactions or over-roasting etc whilst controlling the moisture levels to below a predetermined level. Following this, a coating solution may be applied to the beans to raise the moisture levels to a predetermined desired level and to add the marker.

In some embodiments, the moisture level of the beans may be measured following the initial quench such that the amount of water required to raise the moisture levels is provided in the correct ratio to the marker. Thus, it may be desirable to provide a post-quench moisture content of between 0.5% and 2%, with a final moisture level of between 3% and 5%.

In one example, a blend comprising 50% w/w of washed arabica (Quality Code, QC=031) and 50% unwashed arabica (QC=221) was prepared. Batches of 3 kg of this blend of green coffee beans were roasted isothermally, choosing the air inlet temperature to obtain a final roast colour of 90 CmU (+/−3 CmU) in 360 seconds. The cooling cycle applied at the end of the roast involved a minimal quench of 20 ml and a total cooling time of 150 s, to obtain a low moisture content of the final beans (target 1.5% moisture w/w). Following this, the moisture content was measured and an appropriate coating solution prepared to provide a coffee bean with a desired moisture content and coating weight. In the chosen example, the coating solids were provided at 1% w/w of the finally prepared beans, with a moisture content of 4.2% w/w.

For example, to prepare the coated beans, 200 g of beans with 1.8% moisture were sprayed evenly with 7 g of coating solution containing ˜29% total solids from the various sources given in Table 2. Note that for the addition of minerals as the coating solids, it was established that this concentration of salts would be enough to provide 10-20% of the nutrient reference value, NRV, for the considered mineral per litre of coffee brew (assuming all of the salt is extracted when brewing the coffee), corresponding to 1-2% of NRV per 100 ml serving of the beverage. These amounts are typically a lot less than the nutritional recommended value for a beverage, thereby ensuring that the finally prepared beverage is well within acceptable recommended limits.

TABLE 2
salt solution composition for various salt based quenching agents

| Salt solution | | | Preparation of the | | |
|---|---|---|---|---|---|
| composition | | | coating solutions | | |
| CaCl2 | weight (g) | % (w/w) | Prepare | 50 | g of solution |
| water | 5.00 | | weight | 8.34 | g of CaCl2 |
| solids | 1.00 | 16.7 | Add | 41.66 | g of DI water |
| Total | 6.00 | 100 | Spray | 6.00 | grams of this solution |
| MgCl2 | weight (g) | % (w/w) | Prepare | 50 | g of solution |
| water | 5.00 | | weight | 7.31 | g of MgCl2 |
| solids | 0.86 | 14.6 | Add | 42.69 | g of DI water |
| Total | 5.86 | 100 | Spray | 5.86 | grams of this solution |
| KCl | weight (g) | % (w/w) | Prepare | 50 | g of solution |
| water | 5.00 | | weight | 50.00 | g of KCl |
| solids | 1.34 | 21.2 | Add | 0.00 | g of DI water |
| Total | 6.34 | 100 | Spray | 6.34 | grams of this solution |
| KOH | weight (g) | % (w/w) | Prepare | 50 | g of solution |
| water | 5.00 | | weight | 9.34 | g of KOH |
| solids | 1.15 | 18.7 | Add | 40.66 | g of DI water |
| Total | 6.15 | 100 | Spray | 6.15 | grams of this solution |

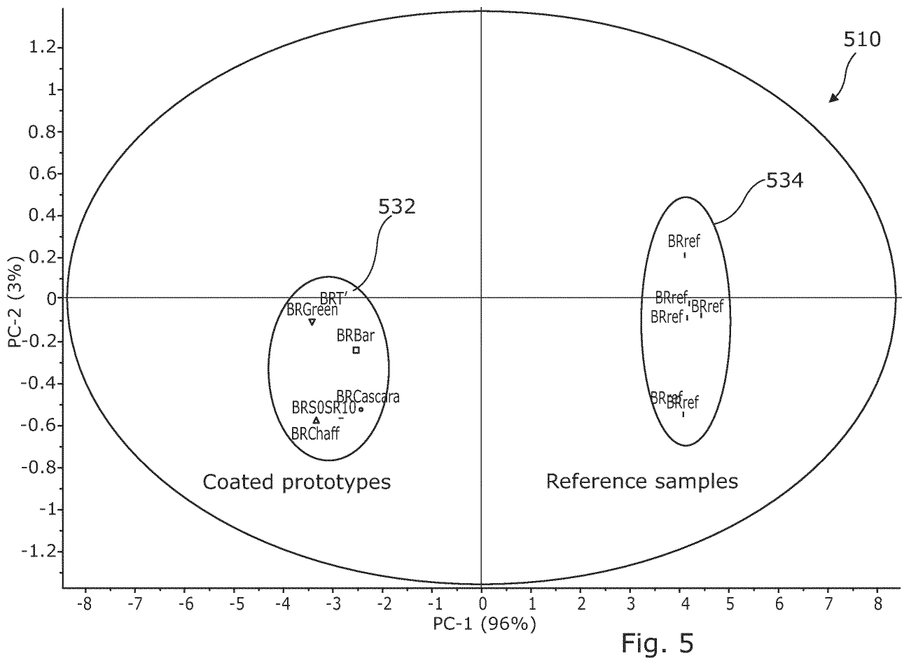

Turning to FIG. 5, there is shown a PCA map 510 for a set of coated samples which include coffee beans coated with organic coffee material. The coated samples 532 form a cluster distinctly different from the reference beans 534 which included no coating. The coated samples include various coatings taken from the coffee-based coatings noted in Table 1. However, how these are distinguished over one another is less relevant compared to the separation from the uncoated reference samples 534.

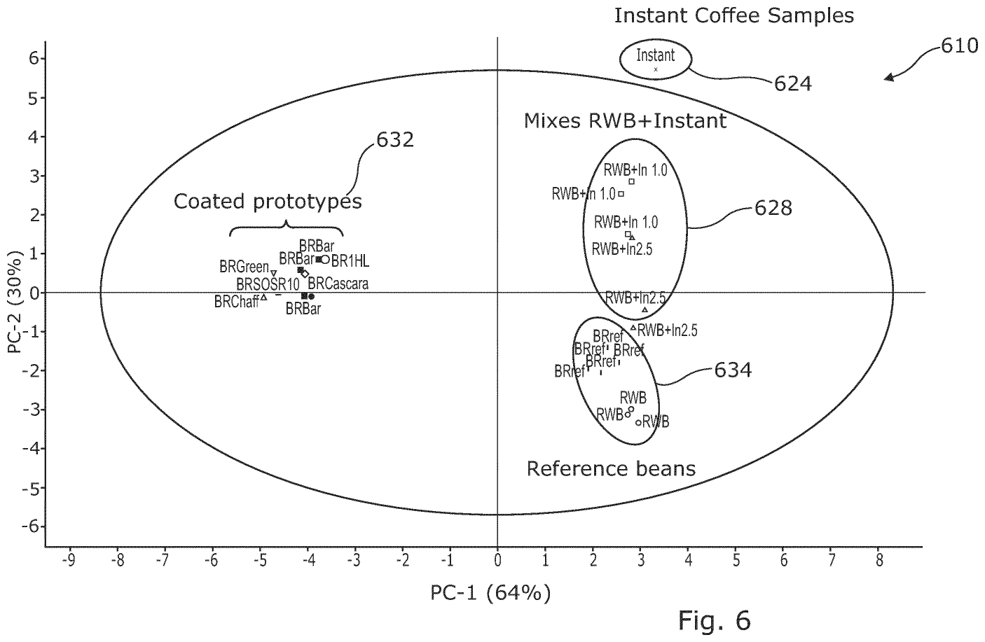

FIG. 6 shows a PCA map 610 for a set of samples comprising the mixed roasted whole bean and instant coffee 628, instant coffee 624, beans coated with an organic coffee-based substance as disclosed herein and reference beans 634 with no marker, again showing the clear differentiation between the groups.

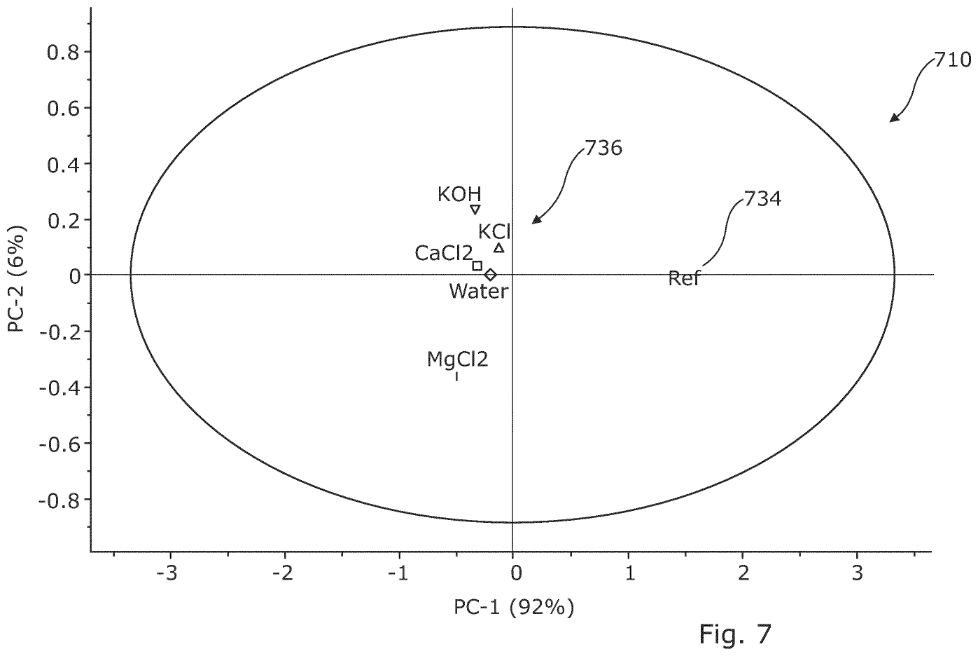

FIG. 7 shows a PCA map 710 with spectra acquired from beans that were coated with mineral salt solutions 736 (as per Table 1) on the left hand side, with uncoated reference beans 734 shown on the right hand side.

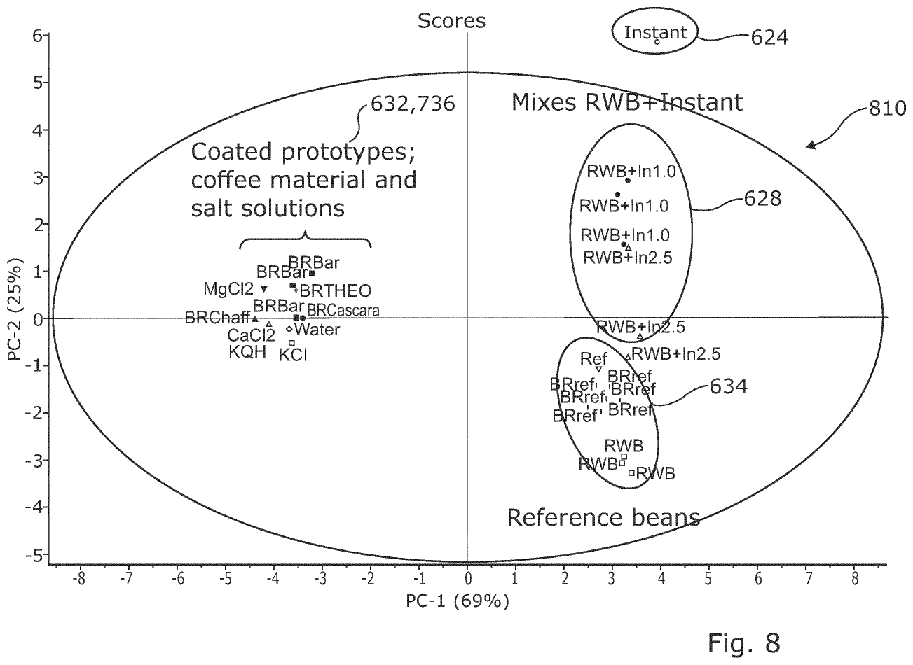

Finally, FIG. 8 shows all of the samples shown in FIGS. 6 and 7 in a common PCA map 810.

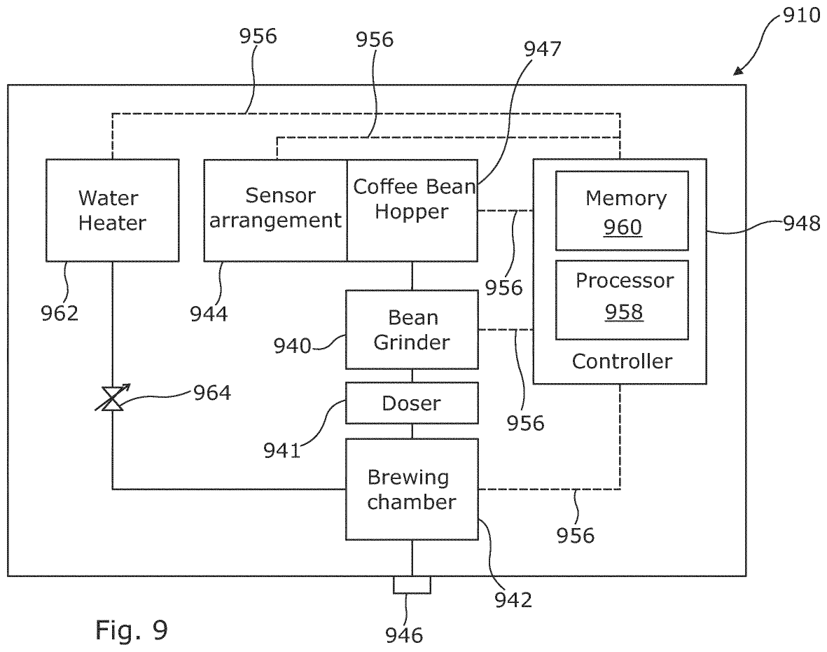

Moving to FIG. 9 there is shown a coffee brewing apparatus 910 which may comprise a bean grinder 940, a doser 941, a coffee brewer 942 and a sensor or sensing arrangement 944. The sensing arrangement 944 may be configured to determine a characteristic of a coffee product which is provided for the purpose of brewing a coffee beverage which is dispensed from a nozzle 946. A coffee product may be stored in a coffee store in the form of a hopper 947 which houses the beans prior to being passed to the bean grinder 940 for grinding.

The coffee brewing apparatus may also comprise a controller 948 which is configured to receive an input from the sensing arrangement 944. The input from the sensing arrangement 944 may relate to at least one characteristic of the coffee product and may be used by the controller 948 to determine one or more brewing parameters of the coffee brewer 942 and/or bean grinder 940. The controller may additionally be configured to control the coffee brewing apparatus 910 to brew a coffee drink using the determined one or more brewing parameters.

### The Sensor Arrangement

The sensor arrangement 944 may be any suitable sensor arrangement which can be used to carry out any of the methods disclosed herein. As such and as described above, the sensor arrangement 944 may be configured to determine a characteristic of the coffee product in order that suitable brewing parameters can be identified.

In some embodiments the characteristic of the coffee product may relate to spectral data of the coffee product, in particular the NIR spectral data. Hence, the sensing arrangement 944 may comprise a spectrometer and may comprise the principal components of a source for emitting the electromagnetic radiation, a detector for receiving the reflected radiation, and a dispersion element, such as a prism or diffraction grating, to allow the intensity of the different wavelengths to be separated and detected. Suitable spectrometers are well known in the art.

The sensing arrangement 944 may be located adjacent to or form part of the hopper 947 such that the source and detector of the sensing arrangement can interface with the coffee product to allow it to be analysed to obtain or determine the characteristic of the coffee product. Hence, for example, the hopper 947 may comprise one or more walls in which an aperture or window is provided to allow the sensing arrangement 944 to illuminate the coffee product and detect the reflected light, thereby obtaining the spectral data.

The position of the sensing arrangement 944 is shown as being on a side wall of the hopper 947 in FIG. 9. However, it will be appreciated that this is not a limitation, and the sensing arrangement 944 may be provided on a base or on a top wall of the hopper 947.

### Controller

The controller 948 may be configured to determine the brewing parameters for the coffee product loaded in the hopper 947 and/or receive the spectral data from the sensing arrangement 944 and/or control the brewing of the coffee beverage using the brewing parameters. As such, the controller 948 may be communicably connected to the sensing arrangement 944, the brewing chamber 942, the bean hopper the grinder 940, the water heater 950 and various valves 952, 954 and any other actuators or sensors for controlling the brewing apparatus 910 via suitable communication buses generically denoted by reference numeral 956. The controller is shown as a separate module in FIG. 9, however, in some embodiments, the controller 948 may form part of the sensing arrangement 944, in which case, the controller 948 may receive an input directly from the sensor of the sensing arrangement 944.

The controller 948 may be configured to carry out any necessary processing of the input received from the sensing arrangement 944 to allow the identification of the brewing parameters to be made. Hence, the controller 948 may be configured to carry out pre-processing and/or processing of the spectral data, as described herein.

As noted, the methods disclosed herein may require the comparison of a characteristic of the coffee product, such as a spectral characteristic, to be compared with one or more characteristics of one or more known coffee products with known associated brewing parameters. As such, the controller 948 may comprise at least one memory on which is stored a plurality of characteristics for a plurality of coffee products having associated brewing parameters so that the controller 948 may, upon determining or obtaining the characteristic of the coffee product in the hopper 947, determine which brewing parameters are required. The store of the plurality of brewing parameters and associated characteristics may be provided in a suitable database and take the form of a simple look-up table. The look-up table may, for example, include a list of spectral features which are associated with particular brewing parameters so that the closes match from amongst the list of spectral features provides the brewing parameters required for the coffee product in the hopper.

In addition to the brewing parameters association with known spectral characteristics, the look-up table may additionally include one or more set of brewing parameters which are default parameters in the case where a characteristic of the coffee product can not be determined.

The controller 948 may comprise any suitable circuitry to cause performance of the methods described herein. The controller 948 may comprise: control circuitry; and/or processor circuitry; and/or at least one application specific integrated circuit (ASIC); and/or at least one field programmable gate array (FPGA); and/or single or multi-processor architectures; and/or sequential/parallel architectures; and/or at least one programmable logic controller (PLC); and/or at least one microprocessor; and/or at least one microcontroller; and/or a central processing unit (CPU); and/or a graphics processing unit (GPU), to perform the methods.

The controller 948 shown in FIG. 9, comprises at least one processor 958 and at least one memory 960. The memory 960 may be configured to store a computer program comprising computer readable instructions that, when read by the processor, causes performance of the methods described herein. The computer program may be software or firmware, or may be a combination of software and firmware.

The processor 958 may be configured to analyse the data received from the sensor arrangement 944 and may be located within the coffee brewing apparatus 910 as shown, or may be located remotely therefrom. As such, the data received from the sensor arrangement 944 may be transmitted to a separate computing device across a suitable communications network. Providing a separate computing device may allow the computing device to be more powerful for the processing of the sensor arrangement data. In doing so, it may allow for the computing power required in the coffee brewing apparatus 910 to be reduced, thereby reducing the cost and size of the machine. Similarly, the memory 960 may be located on the coffee brewing apparatus or remote therefrom.

The memory 960 may be any suitable non-transitory computer readable storage medium, data storage device or devices, and may comprise a hard disk and/or solid state memory (such as flash memory). The memory 948 may be permanent non-removable memory, or may be removable memory (such as a universal serial bus (USB) flash drive or a secure digital card). The memory 948 may include: local memory employed during actual execution of the computer program; bulk storage; and cache memories which provide temporary storage of at least some computer readable or computer usable program code to reduce the number of times code may be retrieved from bulk storage during execution of the code.

The computer program which is executable to carry out any of the methods described herein may be stored on a non-transitory computer readable storage medium. The computer program may be transferred from the non-transitory computer readable storage medium to the memory 960. The non-transitory computer readable storage medium may be, for example, a USB flash drive, a secure digital (SD) card, an optical disc (such as a compact disc (CD), a digital versatile disc (DVD) or a Blu-ray disc). In some examples, the computer program may be transferred to the memory via a wireless signal or via a wired signal.

### Control Panel

The coffee brewing apparatus 910 may comprise a control panel (not shown) which provides an interface with which a user can engage to control the coffee brewing apparatus 910. As such, the control panel may comprise a user interface which is configured to receive inputs from a user and/or to provide a display for providing a user with information. The user interface may, for example, comprise an output display and an input device such as a touch screen input device or one or more buttons to enable a user to select options or operating the machine. The control panel may be provided as part of the controller 910.

### Bean Grinder

The bean grinder 940 may be a conventional bean grinder which receives the whole roasted beans from the hopper 947 and grinds them to a desired grain size for brewing. Where the brewing parameters for a coffee product comprises the grain size of the ground coffee, then the grinder 940 may comprise an adjustable grinding mechanism in which the size of the grind can be adjusted remotely using the controller 948. Thus, the grinder may comprise one or more actuators to adjust the relative spacing of the grinding burrs of the grinding mechanism. Although not shown, there may be a dosing apparatus for controlling the amount of coffee product which is provided to the grinder.

### Coffee Brewer

Coffee brewers of various types are known in the art. The coffee brewer of the present disclosure will typically include a brewing chamber 942 in which hot water and the coffee product are mixed such that the coffee may be extracted from the coffee product and the coffee beverage made for dispensing from the dispensing nozzle 946.

The brewing chamber 942 is configured to receive hot water for brewing from a water heater 962. The water heater may be controllable such that the water is heated by a desired amount in line with the brewing parameters which have been selected for the particular coffee product. The supply line between the water heater 962 and the brewing chamber 942 may comprise an adjustable valve 964 such that the flow rate may be adjusted in line with a desired flow rate which forms part of the brewing parameters. A doser 941 may also be provided between the bean grinder 940 and brewing chamber 942 to allow the amount of coffee deposited in the brewing chamber 942 to be adjusted.

As will be appreciated, the coffee brewing apparatus 910 will include a number of other conventional actuators or sensors which may provide inputs to or be controlled by the controller 910 such that the brewing parameters can be sensed and/or adjusted as required. Hence, for example, the water heater 962 may comprise a heating element and a thermometer, neither of which are shown, but which allow the water to be heated to a required temperature.

### Automation

The coffee brewing apparatus 910 may be an automated apparatus which is configured to sense the coffee product and adjust the brewing parameters without user intervention. Hence, the coffee brewing apparatus 910 may be configured to receive an initialisation input to sense the coffee product and adjust the brewing parameters accordingly. The initialisation input may comprise one or more of: the loading of a coffee product into the hopper or powering up of the coffee beverage apparatus, for example.

### Hopper

The coffee product may be bulk loaded into the hopper 947 such that small quantities are removed for brewing and/or grinding as required. The hopper 947 may be any suitable storage means in which coffee can be temporarily housed prior to being used in the brewing process but will typically comprise an internal or external container or chamber which is externally accessed so as to be loaded with coffee product.

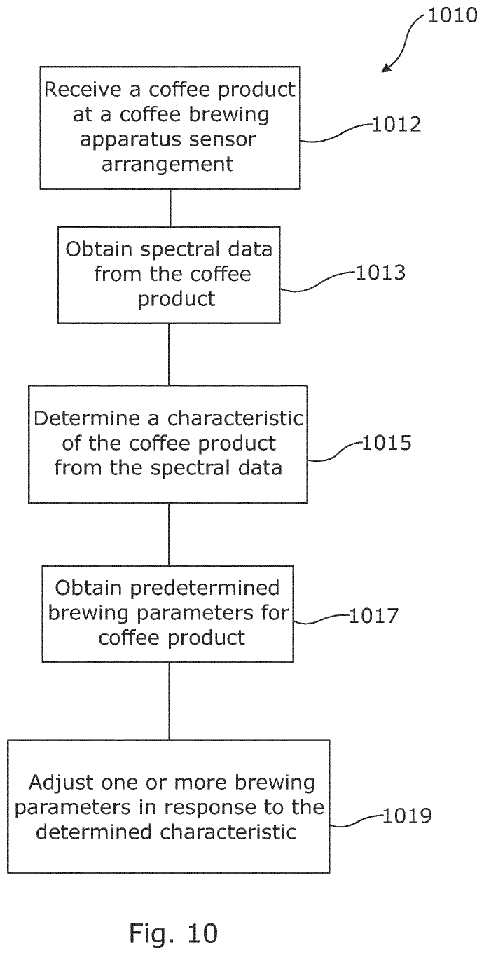

FIG. 10 shows a flow diagram 1010 indicating some method steps for operating the coffee brewing apparatus shown in FIG. 9.

The first step 1012 comprises loading a coffee product into the coffee brewing apparatus 910, e.g. by pouring roasted coffee beans into the hopper 947. In the embodiment of FIG. 9, the sensor arrangement 944 is located adjacent the hopper 947 and the coffee product will be viewable by the sensor arrangement 944 following loading.

The hopper 947 may comprise one or more sensors (not shown) which indicate that the beans have been loaded. Such sensors may include a switch on a hatchway of the hopper 947 which is triggered with the opening or closing of the hatchway, a weight sensor which indicates when the hopper 947 has been loaded with a weight of beans which is sufficient to cover the sensor arrangement 944 to allow the beans to be analysed, or an optical sensor which determines when the hopper 947 has been filled. Alternatively, the control panel or controller 948 may be configured to receive a user input signalling new beans have been loaded and a bean characterisation is required.

Once the need for a bean characterisation has been provided to the controller 948, for example either by a user input or via a signal issued by the aforementioned loading sensors, the controller 948 may send a signal to the sensor arrangement 944 to obtain spectral data 1013. As noted above, spectral data may be obtained by using a spectrometer to illuminate the beans, and detect the reflected light via a diffraction grating to provide the spectral data.

The spectral data is outputted by the sensor arrangement 944 and transmitted to the controller 910 such that a characteristic of the spectral data can be determined 1015. As described herein, the controller 947, e.g. the processor 958 may be configured to process the spectral data such that one or more characteristic which is indicative of the coffee product can be identified and used to determine the required brewing parameters. Hence, once the necessary spectral characteristics have been obtained from the spectral data, a comparison can be made with known spectral data of known coffee products to find a suitable match. Following this, the brewing parameters associated with the known spectral data can be used to brew the coffee product.

The one or more embodiments are described above by way of example only. Many variations are possible without departing from the scope of protection afforded by the appended claims.

1. A method of determining the brewing parameters for preparing a coffee beverage with a coffee brewing apparatus, comprising:
receiving, at a sensing arrangement, a coffee product for brewing a coffee beverage;
obtaining, with the sensing arrangement, a characteristic of the coffee product; and,
using the characteristic to determine one or more brewing parameter for the coffee product.

2. The method of claim 1, wherein the coffee product comprises a marker added or applied to the coffee product, and the characteristic relates to the marker.

3. The method of claim 2, wherein the coffee product comprises coffee beans and the marker comprises one or more of the group consisting of: a surface coating of at least one coffee bean and an instant coffee.

4. The method of claim 3 wherein, when the marker comprises one or more surface coating, the surface coating is organic or a salt.

5. The method of claim 4, wherein the organic coating comprises a coffee-based substance.

6. The method of claim 5, wherein the organic coating comprises one or more from the group comprising: a high temperature extract; a tertiary extract; a cherry extract; a green coffee extract; an instant coffee extract; and silverskin extract.

7. The method of claim 4, wherein the salt comprises calcium dichloride; magnesium dichloride; potassium hydroxide or potassium chloride.

8. The method of any of claims 3 to 7, wherein the surface coating comprises a residue from a quenching agent used to quench the coffee beans following roasting.

9. The method of any preceding claim, wherein the sensing arrangement is configured to obtain spectral data from the coffee product, optionally near infrared spectral data.

10. The method of any preceding claim, wherein the brewing parameters comprise one or more of water temperature, grain size of the ground coffee product, the brew volume, the water flow rate, particle size distribution, type of grinder, compaction amount, volume of coffee dispensed to the coffee brewer and brewing time.

11. The method of any preceding claim, wherein the coffee product is stored within a hopper of the coffee brewing apparatus and the sensing arrangement senses the coffee product whilst in the hopper.

12. The method of any preceding claim, wherein the coffee product comprises a moisture value between 3% and 5% w/w, and the coating comprises between 0.5% and 1.5% w/w solids.

13. The method of any preceding claim, wherein the coffee beverage comprises a solids content of the coating of less than 3% of the nutritional recommended value per 100 ml for the solid.

14. The method of any preceding claim, further comprising:
preparing coffee beans for use in a coffee beverage apparatus, comprising:
roasting green coffee beans in a roaster to provide roasted coffee beans; and,
applying a marker to the roasted coffee beans; and,
providing the coffee beans to a coffee beverage apparatus.

15. The method of claim 14, wherein the marker is provided within a quenching agent.

16. The method of claim 14, further comprising applying a marker to the roasted coffee beans following quenching.

17. The method of claim 16, further comprising determining the moisture level of the roasted coffee beans following quenching;
preparing a water based marking solution having a predetermined amount of marker, wherein the applying the marker to the coffee beans comprises applying the marker solution,
wherein the predetermined amount of marker within the marker solution is determined based on a required final moisture content of the roasted coffee beans and a predetermined amount of required marker residue.

18. The method of any of claims 14 to 17, wherein the coffee product comprises a moisture value between 3% and 5% w/w, and the coating comprises between 0.5% and 1.5% w/w solids.

19. The method of any preceding claim, wherein the prepared coffee comprises a solids content of the coating of less than 3% of the nutritional recommended value per 100 ml for the solid.

20. A coffee brewing apparatus comprising:
a hopper for containing a coffee product;
a sensing arrangement configured to obtain a characteristic of the coffee product; and,
a controller configured to receive an input from the sensing arrangement, the input relating to the characteristic of the coffee product, wherein the controller is configured to determine one or more brewing parameters using the characteristic and control the coffee brewing apparatus to brew a coffee beverage using the one or more brewing parameters.

21. The apparatus of claim 20, wherein the sensing arrangement comprises a near infrared spectrometer.

22. The apparatus of claim 20 or 21, further comprising a coffee grinder and the one or more brewing parameters comprises parameter of the grinder.

23. The apparatus of any of claims 20 to 22, further comprising a brewing chamber and a water heater, wherein the controller is configured to control the water heater to control the water temperature according to the determined brewing parameter and/or the controller is configured to control the water flow rate or brewing time of the coffee product according to the determined brewing parameters.

24. A coffee product for use in a coffee brewing apparatus, the coffee product comprising roasted whole beans and an instant coffee.

25. A method of determining brewing parameters of a coffee product, wherein the coffee product comprises coffee beans and a marker, the method comprising:
obtaining spectroscopic data of the coffee beans and marker;
comparing the spectroscopic data to known spectroscopic data, the known spectroscopic data having associated predetermined brewing parameters; and,
using the predetermined brewing parameters as the brewing parameters for the coffee product when the obtained spectroscopic data and the known spectroscopic data correspond to one another.

26. A method of preparing a coffee product for use in a coffee brewing apparatus, comprising:
roasting the green coffee beans in a roaster to provide roasted coffee beans; and,
applying a quenching agent to the roasted coffee beans to reduce the temperature of the coffee beans;
applying a marker to the coffee beans; and,
providing the coffee beans for a coffee beverage apparatus.
